(19) United States
(12) Patent Application Publication
(10) Pub. No.: US 2003/0027194 A1
(43) Pub. Date: Feb. 6, 2003

(54) Modular assembly of nucleic acid-protein fusion multimers

(76) Inventors: Markus Kurz, Newton, MA (US); Peter Lohse, Weston, MA (US)

Correspondence Address: CLARK & ELBING LLP, 101 FEDERAL STREET, BOSTON, MA, 02110, US

(21) Appl. No.: 10/208,967

(22) Filed: Jul. 31, 2002

Related U.S. Application Data
(60) Provisional application No. 60/309,231, filed on Jul. 31, 2001.

(51) Int. Cl. C12Q 1/68; C12P 21/04; C07K 14/435

(52) U.S. Cl. 435/6; 435/69.7; 435/320.1; 435/325; 530/395

(57) ABSTRACT

Described herein are methods and reagents for generating nucleic acid-protein fusion multimers, and methods for using such fusion molecule multimers to select an interaction between a protein or a peptide and a compound.

### CROSS REFERENCE TO RELATED APPLICATIONS

This application claims the benefit of the filing date of provisional application, U.S. S.No. 60/309,231, filed Jul. 31, 2001, hereby incorporated by reference.

### BACKGROUND OF THE INVENTION

In general, the present invention relates to methods of generating and using nucleic acid-protein fusion multimers.

Certain macromolecules, such as proteins, are known to interact specifically with other molecules based on their three-dimensional shapes and electronic distributions. For example, proteins interact selectively with other proteins, nucleic acids, and small-molecules. The identification of proteins that interact with a target molecule lays the groundwork for the development of compounds to treat diseases and their associated symptoms. However, the discovery of a single drug candidate can require the screening of thousands of compounds, for example, proteins. It is therefore important to be able to screen large numbers of candidates rapidly and efficiently.

### SUMMARY OF THE INVENTION

The present invention features methods for making nucleic acid-protein fusion multimers. Such multimeric fusion complexes can be used for the in vitro selection of multidomain peptides or proteins with desired properties (for example, a desired binding property). A fusion multimer contains a dimerization (or multimerization) domain, which can reside in either the nucleic acid or protein portion of the fusion molecule. In addition, the fusion multimer includes a potential target (or compound) recognition domain in its protein portion, which may be randomized for selection purposes. Once dimerization or multimerization occurs, the recognition domains cooperatively interact with the compound of choice. In certain cases, this may strengthen the dimerization or multimerization of the fusions because of additional binding forces. The compound recognition domains may be, for example, rather simple and unstructured peptide sequences, or may be antibody-like CDR loops or DNA binding motifs, such as zinc finger domains.

Accordingly, in general, in a first aspect, the present invention features novel nucleic acid-protein fusion multimers. One such multimer includes two or more fusion molecules of nucleic acid covalently bound to protein, the nucleic acid of at least one of the fusion molecules encoding the covalently bound protein, wherein the fusion molecules are hybridized to each other through complementary nucleic acid sequences.

Another nucleic acid-protein fusion multimer includes two or more fusion molecules of nucleic acid covalently bound to protein, the fusion molecules not including streptavidin, wherein the fusion molecules are hybridized to each other through complementary nucleic acid sequences.

Another nucleic acid-protein fusion multimer includes two or more fusion molecules of nucleic acid covalently bound at the 3′ end to protein, wherein the fusion molecules are hybridized to each other through complementary nucleic acid sequences.

Another nucleic acid-protein fusion multimer includes two or more fusion molecules of nucleic acid covalently bound to protein through a peptide acceptor, wherein the fusion molecules are hybridized to each other through complementary nucleic acid sequences.

Yet another nucleic acid-protein fusion multimer includes two or more fusion molecules of nucleic acid covalently bound to protein, wherein the covalent bond is not a thiol-maleimide bond, and wherein the fusion molecules are hybridized to each other through complementary nucleic acid sequences.

Another nucleic acid-protein fusion multimer includes (a) two or more fusion molecules of nucleic acid covalently bound to protein, the nucleic acid of at least one of the fusion molecules encoding the covalently bound protein; and (b) an oligonucleotide, wherein a sequence of the nucleic acid of each of the fusion molecules is hybridized to a complementary sequence of the oligonucleotide.

Another nucleic acid-protein fusion multimer includes (a) two or more fusion molecules of nucleic acid covalently bound to protein, the fusion molecules not including streptavidin; and (b) an oligonucleotide, wherein a sequence of the nucleic acid of each of the fusion molecules is hybridized to a complementary sequence of the oligonucleotide.

Another nucleic acid-protein fusion multimer includes (a) two or more fusion molecules of nucleic acid covalently bound at the 3′ end to protein; and (b) an oligonucleotide, wherein a sequence of the nucleic acid of each of the fusion molecules is hybridized to a complementary sequence of the oligonucleotide.

Yet another nucleic acid-protein fusion multimer includes (a) two or more fusion molecules of nucleic acid covalently bound to protein through a peptide acceptor; and (b) an oligonucleotide, wherein a sequence of the nucleic acid of each of the fusion molecules is hybridized to a complementary sequence of the oligonucleotide.

Another nucleic acid-protein fusion multimer includes (a) two or more fusion molecules of nucleic acid covalently bound to protein, wherein the covalent bond is not a thiol-maleimide bond; and (b) an oligonucleotide, wherein a sequence of the nucleic acid of each of the fusion molecules is hybridized to a complementary sequence of the oligonucleotide.

For any of the above multimers that includes an oligonucleotide, that oligonucleotide may have a linear, bi-directional, or branched structure.

The invention further features additional multimers. One such nucleic acid-protein fusion multimer includes (a) two or more fusion molecules of nucleic acid covalently bound to protein; and (b) an oligonucleotide having a bi-directional or branched structure, wherein a sequence of the nucleic acid of each of the fusion molecules is hybridized to a complementary sequence of the oligonucleotide.

Another nucleic acid-protein fusion multimer includes (a) two or more fusion molecules of nucleic acid covalently bound to protein, wherein the nucleic acid of each of the fusion molecules includes a polypurine tract; and (b) an oligonucleotide including at least two polypyrimidine tracts, wherein the polypurine tracts of the fusion molecules are hybridized to the polypyrimidine tracts of the oligonucleotide, and wherein binding of the fusion molecules to the oligonucleotide occurs in opposite directions to form a triple helical structure. In this embodiment, the oligonucleotide may be circular, it may form a clamp-like structure, and/or it may include one or more polyamide nucleic acids.

In preferred embodiments of any of the above multimers of the invention, the fusion molecules may be cross-linked through cross-linking moieties positioned within the nucleic acid of the fusion molecules or the oligonucleotide; and the cross-linking moiety may be psoralen.

The invention further features nucleic acid-protein fusion multimers that include two or more fusion molecules of nucleic acid covalently bound to protein, wherein the protein portions of the fusion molecules each include a multimerization domain, the multimerization domains interacting through non-covalent bond formation.

In preferred embodiments of this aspect of the invention, the multimerization domains interact to form homodimers, heterodimers, trimers, or tetramers; at least two of the multimerization domains include leucine zipper binding regions; the leucine zipper binding regions are derived from a Fos, Jun, or GCN4 leucine zipper binding region; and/or at least one of the multimerization domains includes a tetrazipper binding region.

In further aspects, the invention features a nucleic acid-protein fusion multimer that includes two or more fusion molecules of nucleic acid covalently bound to protein, wherein the protein of each of the fusion molecules includes a multimerization domain that includes a functional group, the functional group of one fusion molecule being linked to the functional group of another fusion molecule through a covalent bond.

In preferred embodiments of this aspect of the invention, the covalent linkage involves an external cross-linking agent; at least one functional group includes an amine or a thiol; at least two functional groups include a thiol and the covalent bond is a disulfide bond; and/or the multimerization domain includes an antibody constant region.

In preferred embodiments of any of the multimers according to the invention, the protein of at least one of the fusion molecules further includes a compound recognition domain. This compound recognition domain may include an antibody variable region and/or a randomized domain. The compound recognition domain may interact with DNA (for example, it may be a zinc finger binding domain). Moreover, any multimer of the invention may include at least one nucleic acid-protein fusion molecule that is an RNA-protein fusion molecule or a DNA-protein fusion molecule.

In yet other embodiments, the invention features an RNA-protein fusion multimer, the multimer including two or more fusion molecules of RNA covalently bound to protein, wherein the fusion molecules are hybridized to each other through complementary nucleic acid sequences.

In preferred embodiments of this aspect of the invention, the RNA of at least one of the fusion molecules encodes the covalently bound protein; the fusion molecules are cross-linked through cross-linking moieties positioned within the RNA of the fusion molecules; and the cross-linking moiety is psoralen.

In a second general aspect, the invention further features methods for preparing the multimers described herein. One such method involves the steps of: (a) providing two or more fusion molecules of nucleic acid covalently bound to protein, the nucleic acid of at least one of the fusion molecules encoding the covalently bound protein; and (b) hybridizing the fusion molecules to each other through complementary nucleic acid sequences, thereby forming a nucleic acid-protein fusion multimer.

Another method involves the steps of: (a) providing two or more fusion molecules of nucleic acid covalently bound to protein, the fusion molecules not including streptavidin; and (b) hybridizing the fusion molecules to each other through complementary nucleic acid sequences, thereby forming a nucleic acid-protein fusion multimer.

Another method for multimer preparation involves the steps of: (a) providing two or more fusion molecules of nucleic acid covalently bound at the 3′ end to protein; and (b) hybridizing the fusion molecules to each other through complementary nucleic acid sequences, thereby forming a nucleic acid-protein fusion multimer.

Another method for multimer preparation involves the steps of: (a) providing two or more fusion molecules of nucleic acid covalently bound to protein through a peptide acceptor; and (b) hybridizing the fusion molecules to each other through complementary nucleic acid sequences, thereby forming a nucleic acid-protein fusion multimer.

Yet another method for multimer preparation involves the steps of: (a) providing two or more fusion molecules of nucleic acid covalently bound to protein, wherein the covalent bond is not a thiol-maleimide bond; and (b) hybridizing the fusion molecules to each other through complementary nucleic acid sequences, thereby forming a nucleic acid-protein fusion multimer.

Another method for multimer preparation involves the steps of: (a) providing two or more fusion molecules of nucleic acid covalently bound to protein, the nucleic acid of at least one of the fusion molecules encoding the covalently bound protein; (b) providing an oligonucleotide, wherein the oligonucleotide includes a plurality of sequences that are complementary to a partner sequence within each of the fusion molecules; and (c) hybridizing the oligonucleotide to each of the fusion molecules, thereby forming a nucleic acid-protein fusion multimer.

Another method for multimer preparation involves the steps of: (a) providing two or more fusion molecules of nucleic acid covalently bound to protein, the fusion molecules not including streptavidin; (b) providing an oligonucleotide, wherein the oligonucleotide includes a plurality of sequences that are complementary to a partner sequence within each of the fusion molecules; and (c) hybridizing the oligonucleotide to each of the fusion molecules, thereby forming a nucleic acid-protein fusion multimer.

Yet another method for multimer preparation involves the steps of: (a) providing two or more fusion molecules of nucleic acid covalently bound at the 3′ end to protein; (b) providing an oligonucleotide, wherein the oligonucleotide includes a plurality of sequences that are complementary to a partner sequence within each of the fusion molecules; and (c) hybridizing the oligonucleotide to each of the fusion molecules, thereby forming a nucleic acid-protein fusion multimer.

Yet another method for multimer preparation involves the steps of: (a) providing two or more fusion molecules of nucleic acid covalently bound to protein through a peptide acceptor, wherein the fusion molecules are hybridized to each other through complementary nucleic acid sequences; (b) providing an oligonucleotide, wherein the oligonucleotide includes a plurality of sequences that are complementary to a partner sequence within each of the fusion molecules; and (c) hybridizing the oligonucleotide to each of the fusion molecules, thereby forming a nucleic acid-protein fusion multimer.

Another method for multimer preparation involves the steps of: (a) providing two or more fusion molecules of nucleic acid covalently bound to protein, wherein the covalent bond is not a thiol-maleimide bond; (b) providing an oligonucleotide, wherein the oligonucleotide includes a plurality of sequences that are complementary to a partner sequence within each of the fusion molecules; and (c) hybridizing the oligonucleotide to each of the fusion molecules, thereby forming a nucleic acid-protein fusion multimer.

For any preparative methods involving an oligonucleotide, that oligonucleotide may have a linear, bi-directional, or branched structure.

Yet another method for multimer preparation according to the invention involves the steps of: (a) providing two or more fusion molecules of nucleic acid covalently bound to protein; (b) providing an oligonucleotide having a bi-directional or branched structure, wherein the oligonucleotide includes a plurality of sequences that are complementary to a partner sequence within each of the fusion molecules; and (c) hybridizing the oligonucleotide to each of the fusion molecules, thereby forming a nucleic acid-protein fusion multimer.

Another method for multimer preparation involves the steps of: (a) providing two or more fusion molecules of nucleic acid covalently bound to protein, wherein the nucleic acid of each of the fusion molecules includes a polypurine tract; (b) providing an oligonucleotide including at least two polypyrimidine tracts; and (c) hybridizing the polypurine tracts to the polypyrimidine tracts, wherein binding of the fusion molecules to the oligonucleotide occurs in opposite directions to form a triple helical structure, thereby forming a nucleic acid-protein multimer.

In this embodiment of the invention, the oligonucleotide may be circular, may form a clamp-like structure, and/or may include one or more polyamide nucleic acids.

In any preparative technique according to the invention, the method may further involve cross-linking the fusion molecules to each other or to the oliogonucleotide. This cross-linking may be carried out by functionalizing the fusion molecules or the oligonucleotide with psoralen and irradiating the nucleic acid-protein fusion multimer.

Yet another inventive method for multimer preparation involves the steps of: (a) providing two or more fusion molecules of nucleic acid covalently bound to protein, wherein the protein portions of the fusion molecules each include a multimerization domain; and (b) combining the fusion molecules under conditions that allow non-covalent interactions between the multimerization domains, thereby forming a nucleic acid-protein fusion multimer.

In preferred embodiments of this aspect of the invention, the multimerization domains interact to form homodimers, heterodimers, trimers, or tetramers; at least two of the multimerization domains include leucine zipper binding regions; the leucine zipper binding regions are derived from a Fos, Jun, or GCN4 leucine zipper binding region; and/or at least one of the multimerization domains includes a tetrazipper binding region.

In yet another method according to the invention, a nucleic acid-protein fusion multimer is prepared by the steps of: (a) providing two or more fusion molecules of nucleic acid covalently bound to protein, wherein the protein of each of the fusion molecules includes a multimerization domain that includes a functional group; and (b) combining the fusion molecules under conditions that allow the functional group of one fusion molecule to be linked to the functional group of another fusion molecule through a covalent bond, thereby forming a nucleic acid-protein fusion multimer.

In preferred embodiments of this method, the covalent linkage involves an external cross-linking agent; at least one functional group includes an amine or a thiol; at least two functional groups include a thiol and the covalent bond is a disulfide bond; and/or the multimerization domain includes an antibody constant region.

In preferred embodiments of any of the preparative methods of the invention, the protein of at least one of the fusion molecules further includes a compound recognition domain; the compound recognition domain includes an antibody variable region; and/or the compound recognition domain includes a randomized domain. Such a compound recognition domain may interact with DNA (for example, it may be a zinc finger binding domain. In addition, for any preparative method described herein, at least one of the nucleic acid-protein fusion molecules may be an RNA-protein fusion molecule or a DNA-protein fusion molecule.

In yet another method according to the invention, an RNA-protein fusion multimer is prepared by the steps including: (a) providing two or more fusion molecules of RNA covalently bound to protein; and (b) hybridizing the fusion molecules to each other through complementary nucleic acid sequences, thereby forming an RNA-protein fusion multimer.

In a third general aspect, the invention further features a method of selecting a protein that interacts with a compound, the method involving the steps of: (a) providing a population of candidate nucleic acid-protein fusion multimers, the multimers including two or more hybridized or covalently bound fusion molecules of nucleic acid covalently bound to protein; (b) providing a compound; (c) contacting the compound with the population of candidate nucleic acid-protein fusion multimers under conditions that allow an interaction between the compound and the candidate nucleic acid-protein fusion multimers; and (d) selecting a nucleic acid-protein fusion multimer that interacts with the compound, thereby selecting a protein that interacts with the compound.

In one preferred embodiment, the compound is immobilized on a column.

In another preferred embodiment, the method further involves the steps of: (e) dissociating the nucleic acid-protein fusion multimers that do not interact with the compound; (f) recombining the dissociated nucleic acid-protein fusion multimers; (g) contacting the compound with the recombined nucleic acid-protein fusion multimers; and (h) selecting a recombined nucleic acid-protein fusion multimer that interacts with the compound, thereby selecting a protein that interacts with the compound. In this method, the dissociation and recombination of the nucleic acid-protein fusion multimers that do not interact with the compound may occur through heating and cooling of the fusion molecules or through denaturation by a reduction reaction, followed by recombination under oxidative conditions. Steps (e) through (h) may be repeated any number of times.

In another preferred approach of the selection methods, the population, in step (a), is maintained under equilibrium conditions, whereby the individual fusion molecules of the nucleic acid-protein fusion multimers rapidly dissociate and associate with other individual fusion molecules, thereby forming new nucleic acid-protein fusion multimers.

In yet another preferred embodiment, the method having steps (a)-(d) described above further involves the steps of: (e) amplifying the nucleic acids of the nucleic acid-protein fusion multimers selected in step (d); (f) generating, from the amplified nucleic acids, fusion molecules of nucleic acid covalently bound to protein; (g) generating from those fusion molecules a second population of nucleic acid-protein fusion multimers; and (h) repeating steps (b) through (d).

In other preferred embodiments of the selection methods, the compound interacts with the nucleic acid-protein fusion multimer in solution and is subsequently immmobilized on a solid phase; the compound is detectably labeled; the compound is detectably labeled with biotin and the solid support is a streptavidin resin; and/or at least one of the fusion molecules is an RNA-protein fusion molecule or a DNA-protein fusion molecule.

As used herein, by “nucleic acid-protein fusion molecule” is meant a molecule comprising a nucleic acid covalently bound directly or indirectly to a protein. This molecule may also include additional components, for example, a non-nucleosidic spacer or psoralen. The nucleic acid molecule may be an RNA or DNA molecule, or may include RNA or DNA analogs at one or more positions in the sequence. Alternatively, the nucleic acid portion of the fusion may be partially or wholly composed of a PNA sequence. The “protein” portion of the fusion is composed of two or more naturally occurring or modified amino acids joined by one or more peptide bonds. “Protein” and “peptide” are used interchangeably herein. Exemplary RNA-protein fusion molecules are described, for example, by Roberts and Szostak (Proc. Natl. Acad. Sci. USA 94:12297-302, 1997); Szostak et al. (WO 98/31700; WO 00/47775); and Gold et al. (U.S. Pat. No. 5,843,701). Exemplary DNA-protein fusion molecules are described, for example, in U.S. Pat. No. 6,416,950; WO 00/32823, all of which are hereby incorporated by reference.

By “nucleic acid-protein fusion multimer” is meant two or more identical or different nucleic acid-protein fusions bound together covalently or non-covalently.

By “functionalize” is meant to chemically modify a molecule in a manner that results in the attachment of a functional group or moiety. For example, a nucleic acid-protein fusion molecule can be functionalized with a cross-linking moiety such as psoralen, an azido compound, or a sulfur-containing nucleoside.

By “hybridize” is meant to associate complementary nucleic acid sequences with one another. Standard hybridization techniques are known in the art (see, for example, Ausubel et al, Current Protocols in Molecular Biology, John Wiley & Sons, New York, N.Y., 1998). Preferably, the nucleic acid sequences hybridize under low temperature (for example, less than or equal to room temperature), medium to high ionic strength buffer (for example, in a buffer containing 100 mM or greater salt concentration), and neutral pH (for example, pH 7-8).

By a “multimerization domain” of a nucleic acid-protein fusion molecule is meant a portion of a nucleic acid-protein fusion molecule that binds that fusion molecule to one or more additional nucleic acid-protein fusion molecules to form a nucleic acid-protein fusion multimer. Binding may be covalent or non-covalent. The multimerization domain may be located in the protein portion of the fusion molecule, where multimerization of the fusion molecules occurs through interaction of the protein multimerization domains. Examples of protein multimerization domains include, but are not limited to, domains that are functionalized such that they interact with one another, domains capable of forming homodimers, heterodimers, trimers, or tetramers (for example leucine zipper binding regions or tetrazipper binding regions), and antibody constant regions. Alternatively, the multimerization domain may be located in the nucleic acid portion of the fusion molecule, where multimerization of the fusion molecules occurs, for example, through hybridization. “Multimerization domains” also may be located on an external oligonucleotide, in which case the domains hybridize with the nucleic acid portions of fusion molecules to form a multimer.

By a “compound recognition domain” is meant a portion of a nucleic acid-protein fusion molecule that facilitates interaction of that molecule (or its associated complex) with a compound (e.g., by binding or by catalyzing a binding event). The compound recognition domain may be located in the protein portion of the fusion molecule and may include, for example, a randomized amino acid sequence, a naturally occurring or optimized domain capable of interacting with DNA (for example, a zinc finger binding domain), or an antibody variable region. Compound recognition domains may facilitate compound interactions alone or preferably in association with the compound recognition domains of other associated fusion molecules in a complex.

By a “peptide acceptor” is meant any molecule capable of being added to the C-terminus of a growing protein chain by the catalytic activity of the ribosomal peptidyl transferase function. Typically, such molecules contain (i) a nucleotide or nucleotide-like moiety (for example, adenosine or an adenosine analog (dimethylation at the N-6 amino position is acceptable)), (ii) an amino acid or amino acid-like moiety (for example, any of the 20 D- or L-amino acids or any amino acid analog thereof (for example, O-methyl tyrosine or any of the analogs described by Ellman et al., Meth. Enzymol. 202:301, 1991), and (iii) a linkage between the two (for example, an ester, amide, or ketone linkage at the 3′ position or, less preferably, the 2′ position); preferably, this linkage does not significantly perturb the pucker of the ring from the natural ribonucleotide conformation. Peptide acceptors may also possess a nucleophile, which may be, without limitation, an amino group, a hydroxyl group, or a sulfhydryl group. In addition, peptide acceptors may be composed of nucleotide mimetics, amino acid mimetics, or mimetics of the combined nucleotide-amino acid structure. Exemplary peptide acceptors are described, for example, in Szostak et al., WO 98/31700.

By “selecting,” as it applies to proteins, is meant identifying, detecting, or substantially isolating a protein in a nucleic acid-protein fusion multimer that interacts with a compound. A protein may be selected, for example, by immobilizing a compound on a column, running a solution containing candidate nucleic acid-protein fusion multimers through the column, binding the fusion multimers of interest to the column by affinity interactions with the compound, removing non-specific fusions from the column, and eluting the multimers that bound to the compound. The nucleic acid-protein fusion multimers contained in the eluate are those that contain proteins that are selected. Thus, by “selecting” a nucleic acid-protein multimer that interacts with a compound, one can “select” a protein that interacts with the compound.

By a “compound” or “ligand” is meant a chemical molecule, be it naturally-occurring or artificially-derived, and includes, for example, peptides, proteins, synthetic organic molecules, naturally-occurring organic molecules, nucleic acid molecules, and components thereof.

The advantages of in vitro selection of proteins or peptides using nucleic acid-protein fusion multimers are many. For example, multimerization allows the preparation of candidate pools with increased complexity by allowing the combination of identical copies of the same nucleic acid-protein fusion molecule with different partners. In addition, equilibrium association and dissociation of the pool members can allow dynamic recombination, thereby further increasing the pool complexity by repeatedly producing new combinations of nucleic acid-protein fusion multimers.

Furthermore, in contrast to certain gene recombination methods, the present invention provides a method for domain shuffling on the phenotype level. Dynamic recombination of nucleic acid-protein fusion molecules can take place at any time during the course of an in vitro selection experiment. This potential for inherent steady recombination of nucleic acid-protein fusion molecules, as described below, can offer advantages for the in vitro selection of proteins with novel properties.

Other features and advantages of the invention will be apparent from the following detailed description, and from the claims.

### DETAILED DESCRIPTION

The drawings will first briefly be described.

### BRIEF DESCRIPTION OF THE DRAWINGS

FIG. 1A is a schematic representation of the heterodimerization of nucleic acid-protein fusion molecules through duplex formation of the nucleic acid portion of the fusion molecule or the linker DNA.

FIG. 1B is a schematic representation of the homodimerization of nucleic acid-protein fusion molecules through duplex formation of the nucleic acid portion of the fusion molecule or the linker DNA. Homodimers are formed when the nucleic acid portions of the fusion molecules or the DNA linkers contain palindromic sequences.

FIG. 2A is a schematic representation of the multimerization of nucleic acid-protein fusion molecules through ternary complex formation with a connector oligonucleotide. The connector oligonucleotide may be a linear sequence that simultaneously binds to more than one fusion molecule by Watson-Crick base pairing.

FIG. 2B is a schematic representation of the multimerization of nucleic acid-protein fusion molecules through ternary complex formation with a connector oligonucleotide. The connector oligonucleotide may be a bi-directional oligonucleotide.

FIG. 2C is a schematic representation of the multimerization of nucleic acid-protein fusion molecules through ternary complex formation with a connector oligonucleotide. The connector oligonucleotide may be a branched oligonucleotide.

FIG. 3A is a schematic representation of the multimerization of nucleic acid-protein fusion molecules through ternary complex formation with a connector oligonucleotide. The connector oligonucleotide may be a circular triplex-forming oligonucleotide. Target sequences on the nucleic acid-protein fusion molecules are designed to contain polypurine tracts that bind to two interconnected polypyrimidine tracts contained in the oligonucleotide. Each polypyrimidine tract binds one nucleic acid-protein fusion molecule through antiparallel Watson-Crick base pairing, while binding the other nucleic acid-protein fusion molecule in a parallel Hoogsteen mode.

FIG. 3B is a schematic representation of the multimerization of nucleic acid-protein fusion molecules through ternary complex formation with a connector oligonucleotide. The connector oligonucleotide may be a triplex-forming oligonucleotide having a clamp-like structure. Target sequences on the nucleic acid-protein fusion molecules are designed to contain polypurine tracts that bind to two interconnected polypyrimidine tracts contained in the oligonucleotide. Each polypyrimidine tract binds one nucleic acid-protein fusion molecule through antiparallel Watson-Crick base pairing, while binding the other nucleic acid-protein fusion molecule in a parallel Hoogsteen mode.

FIG. 4A is a schematic representation of the protein-based multimerization of nucleic acid-protein fusion molecules. In their protein portions, the fusion molecules contain multimerization domains that bind one another.

FIG. 4B is a schematic representation of the protein-based multimerization of nucleic acid-protein fusion molecules occurring when the protein portion of the fusion molecules includes an antibody Fab fragment. The protein portions of the fusion molecules contain constant regions C_{H} and C_{L} (i.e., multimerization domains) which mediate association followed by disulfide bond formation, allowing the randomized variable regions V_{H} and V_{L} (i.e., compound recognition domains) to be correctly positioned for recognition and binding of potential antigens.

FIG. 4C is a schematic representation of the protein-based multimerization of nucleic acid-protein fusion molecules occurring when the protein portion of the nucleic acid-protein fusion molecule contains a DNA binding domain (i.e., compound recognition domain), and a double stranded DNA target molecule is provided. The dimer is formed by the interaction of the DNA binding domain, for example, a zinc finger domain, with the DNA target molecule. In this format, the multimerization and compound recognition domains may overlap. Alternatively, the multimerization domain and compound recognition domain may differ. For example, candidate DNA binding complexes may exploit a leucine zipper domain for multimerization purposes and a zinc finger domain (or randomized or mutagenized domain) for DNA recognition purposes.

FIG. 5 is a schematic representation illustrating how the complexity of nucleic acid-protein fusion multimer libraries can be increased through multiple nucleic acid-protein fusion libraries. Two independent sub-libraries (e.g., sub-libraries A and B) are generated, each library containing multiple copies of each nucleic acid-protein fusion molecule. The sub-libraries are then combined, and dimerization of the fusion molecules of the two different sub-libraries occurs. During the dimerization step, each particular nucleic acid-protein fusion molecule from sub-library A will be combined with a different nucleic acid-protein fusion molecule from sub-library B, resulting in a library of unique members.

FIG. 6 is a schematic representation illustrating how library complexity is increased through repeated recombination during the selection step. A library of nucleic acid-protein fusion molecules is generated and multimerized. The library is then passed over a column containing the desired immobilized affinity ligand. Those nucleic acid-protein fusion multimers that bind the ligand are retained on the column, while the remaining nucleic acid-protein fusion multimers are recovered in the eluate. In the next step, the multimers are dissociated, and then recombined to form new multimers. These newly formed multimerized nucleic acid-protein fusion complexes are then passed over the column.

FIG. 7 is a schematic representation illustrating how the complexity of a nucleic acid-protein fusion multimer library can be increased through dynamic recombination. The nucleic acid-protein fusion complexes of this library dimerize through weak, non-covalent interactions. In an equilibrium state, those molecules rapidly associate and dissociate, thereby constantly creating new multimeric species. Suitable complexes then jointly bind to a ligand, which increases the overall complex stability and removes this complex from equilibrium to separate the selected nucleic acid-protein fusion complexes from unbound species.

FIG. 8 is a schematic representation illustrating how the diversity of a nucleic acid-protein fusion multimer library is repeatedly generated when proceeding from one molecular generation to the next. In generation n, specific nucleic acid-protein fusion molecule multimers are selected for by binding a target. As part of the preparation of the generation n+1 for the next round of selection, those fusion molecules that were selected for in the previous step as binding a target are generated again and added to the n+1 generation of nucleic acid-protein fusion multimers to provide additional diversity and to combine advantageous binding features to shape tighter binders.

Described herein are methods for making nucleic acid-protein fusion multimers and using such fusion complexes to select desired proteins and peptides in the form of nucleic acid-protein fusion molecules. Techniques for carrying out each method of the invention are now described in detail, using particular examples. These examples are provided for the purpose of illustrating the invention, and should not be construed as limiting.

### EXAMPLE 1

### Formation of Nucleic Acid-Protein Fusion Molecules

As described above, nucleic acid-protein fusion molecules useful in the invention may include any nucleic acid or nucleic acid analog covalently bonded to any naturally occurring or modified peptide sequences. To generate RNA-protein fusions in which the RNA encodes the associated protein, individual RNA sequences (or a plurality of sequences) may be translated in vitro, and fusions formed, for example, according to the methods of Roberts and Szostak (supra) and Szostak et al. (supra). The RNA for the in vitro translation reaction may be generated by any standard approach, including normal cellular synthesis, recombinant techniques, chemical synthesis, and enzymatic synthesis (e.g., in vitro transcription using, for example, T7 RNA polymerase), and useful RNA libraries according to the invention include, without limitation, cellular RNA, mRNA libraries, and random synthetic RNA libraries. A peptide acceptor in the method of Szostak et al. (supra) (for example, puromycin) is bonded to the RNA through a nucleic acid or nucleic acid analog linker. Almost any spacer unit may be used to bind the peptide acceptor to the RNA. For example, spacer units provided by Glen Research (Sterling, Va.) may be utilized, particularly, triethylene glycol phosphate (Spacer 9), hexaethylene glycol phosphate (Spacer 18), propylene phosphate (Spacer C3), and dodecamethylene phosphate (Spacer C 12) spacers. Additional exemplary nucleic acid analogs include, for example, a polyamide nucleic acid (PNA; Nielsen et al., Science 254:1497, 1991), a P-RNA (Krishnamurthy, Angew. Chem. 35:1537, 1996), or a 3′N phosphoramidate (Gryaznov and Letsinger, Nucleic Acids Res. 20:3403, 1992). Such peptide acceptor molecules may be generated by any standard technique, for example, the techniques described in Roberts and Szostak (supra) and Szostak et al. (supra).

An RNA-protein fusion molecule preferably consists of an mRNA molecule that includes a translation initiation sequence and a start codon operably linked to a candidate protein coding sequence and a peptide acceptor at the 3′ end of the candidate protein coding sequence. A DNA or RNase resistant nucleic acid analog linker sequence is included between the end of the message and the peptide acceptor. If desired, groups or collections of RNA sequences, for example, from a particular source or of a given type, may be translated together in a single reaction mixture according to the same general procedure.

DNA-protein fusion molecules may also be used to carry out the present invention. Such DNA-protein fusion molecules are similar to the above-described RNA-protein fusion molecules, except that the DNA, for example, a cDNA is covalently attached to the protein portion of the fusion molecule. Preferably the DNA contains the genetic information for the protein to which it is bonded. DNA-protein fusion molecules may be generated, for example, according to the methods provided by U.S. Pat. No. 6,416,950; WO 00/32823.

For selection purposes, the fusion molecules so produced preferably include a protein domain having a candidate compound recognition domain that facilitates interaction with a target compound, either alone or in association with the compound recognition domains from other associated fusions in a fusion multimer. In addition, either the nucleic acid or protein portion of the fusion molecule includes a multimerization domain that facilitates the formation of multimeric complexes with other fusion molecules in a population.

### EXAMPLE 2

### Formation of a Nucleic Acid-Protein Fusion Multimer through Direct Hybridization of the Nucleic Acid Portions of the Nucleic Acid-Protein Fusion Molecules

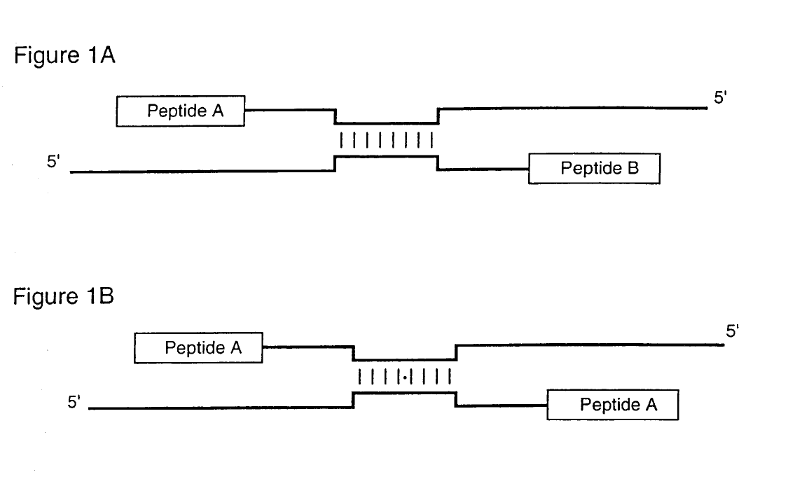

Nucleic acid-protein fusion molecules can be multimerized, for example, through duplex formation of nucleic acid portions located in either the RNA or linker DNA portions of RNA-protein fusion molecules or located in DNA-protein fusion molecules. As shown in FIG. 1A, the base-pairing regions can be specifically designed to allow the formation of heterodimers (for example, A-B heterodimers) or pools of fusion molecules (for example, A_{a-z}; B_{a-z}). Alternatively, the use of palindromic sequences in the DNA portion of the DNA-protein fusion molecule or in the DNA linker allows the formation of homodimers (A-A; see FIG. 1B). This strategy is not restricted to dimerization events, as multiple nucleic acid-protein fusion molecules can be connected by direct hybridization. Preferably, multimerization domains in the nucleic acid sequences hybridize under low temperature (for example, less than or equal to room temperature), medium to high ionic strength buffer (for example, in a buffer containing 100 mM or greater salt concentration), and neutral pH (for example, pH 7-8). In addition, the hybridizing sequence length for each nucleic acid portion of the fusion molecule is preferably between 15 and 25 nucleotides.

The direct hybridization of nucleic acid-protein fusion molecules to one another requires careful purification of the nucleic acid-protein fusion molecules from unfused nucleic acids or DNA linkers. Contaminating unfused nucleic acids or DNA linkers would also be subject to hybridization with nucleic acid-protein fusion molecules and therefore interfere with dimerization. In addition, the careful choice of length and sequence of the hybridization domains permits the tailoring of the thermodynamic stability of the multimeric fusion molecule complex. This is especially important in applications where equilibrium association and dissociation of the fusion molecules are crucial, as described below. The nucleic acid-protein fusion molecules can be purified according to the methods of Roberts and Szostak (supra).

### EXAMPLE 3

### Formation of a Nucleic Acid-Protein Fusion Multimer through Ternary Complex Formation Using a Connector Oligonucleotide

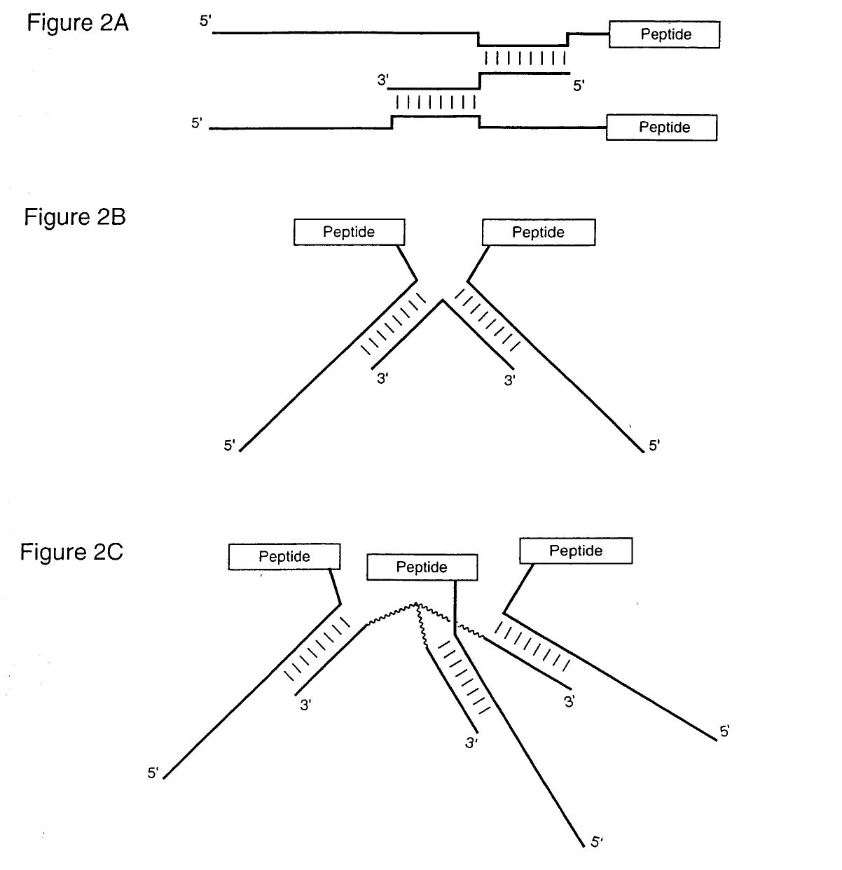

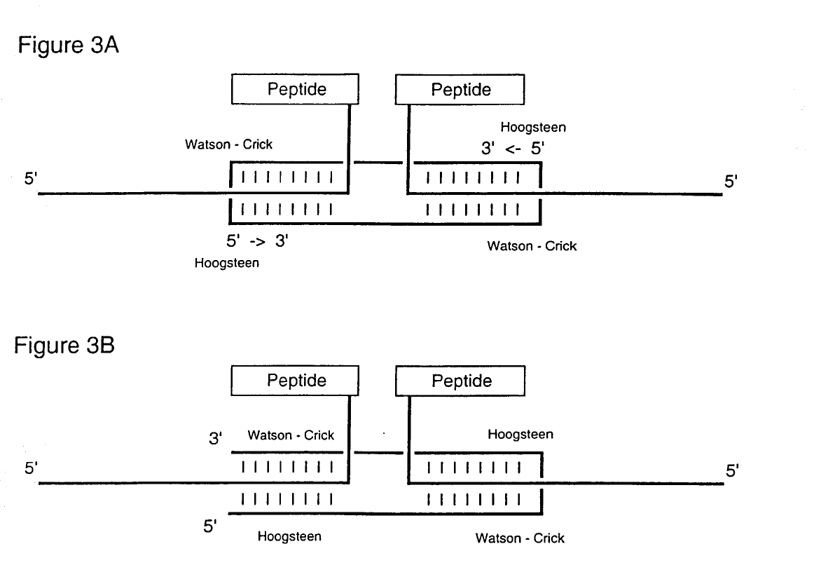

The multimerization of individual nucleic acid-protein fusion molecules can also be mediated by an external oligonucleotide. For example, multimerization domains in a simple linear oligonucleotide sequence can be utilized which simultaneously bind to the multiple domains in more than one nucleic acid-protein fusion molecule through Watson-Crick base pairing (FIG. 2A). Heteromultimeric or homomultimeric nucleic acid-protein fusion molecules may be generated by designing an oligonucleotide that contains sequences that hybridize to a sequence contained in each of the desired members of the multimeric fusion molecule.

In a similar approach, nucleic acid-protein fusion multimers can be formed by hybridizing the nucleic acid multimerization domain of each fusion molecule to multimerization domains in a bi-directional template oligonucleotide (FIG. 2B). If desired, the protein portions of the nucleic acid-protein fusion may be positioned near each other, by designing the template oligonucleotide to hybridize to the 3′ end of the nucleic acid portion of the fusion molecule, or, more preferably, to nucleic acid sequences adjacent to the puromycin at the 3′-end of the linker portions of the fusion molecules.

The required reversal of sequence polarity of the bi-directional oligonucleotides to be used in this method can be easily introduced by standard DNA synthesis of one half of the template molecule, followed by synthesis of the second half using 5′-phosphoramidites (Glen Research).

Preferably the length of the hybridizing sequence of the nucleic acid-protein fusion molecule and the oligonucleotide is between 15 and 25 nucleotides for each fusion molecule, resulting in a total hybridization sequence length of 30 to 50 nucleotides if the multimeric fusion molecule contains two fusion molecules. Optimal hybridization conditions include low temperature (for example, less than or equal to room temperature), medium to high ionic strength buffer (for example, in a buffer containing 100 mM or greater salt concentration), and neutral pH (for example, pH 7-8).

### EXAMPLE 4

### Formation of a Nucleic Acid-Protein Fusion Multimer through Ternary Complex Formation Using a Branched Connector Oligonucleotide

Nucleic acid-protein fusion multimers can also be generated by hybridizing individual fusion molecules to a branched connector oligonucleotide (FIG. 2C). For example, a branching amidite (reagents available from Clontech, Palo Alto, Calif.) may be used to synthesize a branched oligonucleotide, wherein each branch of the oligonucleotide contains a multimerization domain that hybridizes to a multimerization domain in the nucleic acid portion or the linker of one or more individual fusion molecules. Branched connector oligonucleotides consisting of multiple branching points may also be designed and used to form multimeric nucleic acid-protein fusion molecules. The optimal length of the hybridizing sequence is 15 to 25 nucleotides for each fusion molecule of the multimeric nucleic acid-protein fusion molecule. The optimal hybridization conditions are as in Example 3.

### EXAMPLE 5

### Formation of a Nucleic Acid-Protein Fusion Multimer Using a Circular Triplex-Forming Oligonucleotide

Nucleic acid-protein fusion multimers can also be formed by using circular triplex-forming oligonucleotides (TFO) as templates to which individual fusion molecules bind (Selvasekaran and Turnbull, Nucleic Acids Res. 27:624, 1999). Target sequences (multimerization domains) on the nucleic acid-protein fusion molecules, preferably in the linker DNA, can be designed to consist of polypurine tracts. These tracts bind to polypyrimidine tracts contained within the TFO; each polypyrimidine tract in the TFO targets one nucleic acid-protein fusion molecule through antiparallel Watson-Crick base-pairing while binding the second nucleic acid-protein fusion molecule in a parallel Hoogsteen mode. Thus, the two nucleic acid-protein fusion molecules can be bound in opposite directions, allowing the peptide moieties to be positioned next to each other. Preferably the length of each polypurine and polypyrimidine tract is between 15 and 25 nucleotides. Hybridization of the polypurine and polypyrimidine tracts occurs at low temperatures using a medium to high ionic strength buffer containing multivalent cations (e.g., Mg²⁺, spermine, or spermidine) at a slightly acid pH (e.g., pH approximately 5 to 6). In addition, more than two nucleic acid-protein fusion molecules may be bound together with one triplex-forming oligonucleotide template.

For this approach, polyamide nucleic acids (PNAs) would be a suitable alternative to conventional DNA, because of their favorable behavior in triplex formation (Uhlman et al., Angew. Chem. Int. Ed. 37:2796-2823, 1998).

### EXAMPLE 6

### Formation of a Nucleic Acid-Protein Fusion Multimer Using a Clamp-Like Triplex-Forming Oligonucleotide

Nucleic acid-protein fusion multimers can also be formed using a method similar to that described above. In this method, multimerization domains in individual nucleic acid-protein fusion molecules are hybridized to multimerization domains in a TFO oligonucleotide that has a clamp-like structure. As in the above Example, polyamide nucleic acids (PNAs) may be used in the synthesis of such TFOs. Preferably the polypurine and polypyrimidine tracts are between 10 and 15 nucleotides in length, with hybridization occurring at low temperature (for example, less than or equal to room temperature), using a low to medium ionic strength buffer (for example, in a buffer containing less than or equal to 50 mM) at a slightly acidic pH (e.g., pH approximately 5 to 6).

It is important to point out that the purity of the nucleic acid-protein fusion molecules should be high for each of the above-described methods of generating multimeric fusion complexes, as contaminating unfused nucleic acids or DNA linkers may hybridize with the nucleic acid-protein fusion molecules, thereby interfering with the multimerization process.

### EXAMPLE 7

### Formation of a Nucleic Acid-Protein Fusion Multimer through Covalent Bond Formation between the Nucleic Acid Portions of Fusion Molecules

If desired, an enhancement of the methods of any of the above Examples may be implemented by adding stability to the hybridized fusion molecules and oligonucleotides through the formation of covalent bonds between those nucleobases involved in the hybridization. For example, oligonucleotide connectors functionalized to carry psoralen moieties allow the introduction of cross-links to complementary nucleic acids in the fusion molecule upon irradiation with long wave UV-light. Such psoralen moieties can be positioned at strategic positions within (Pieles and Englisch, Nucleic Acids Res. 17:285, 1989) or adjacent to (Pieles et al., Nucleic Acids Res. 17:8967, 1989) an oligonucleotide to be base paired with the fusion molecule(s).

### EXAMPLE 8

### Covalent Bond Formation between the Peptide Portions of Nucleic Acid-Protein Fusion Molecules

Nucleic acid-protein fusion multimers can also be formed through covalent interactions between the protein portions of individual fusion molecules. For example, suitable functional groups (e.g., —NH₂ and —SH) that are present in the multimerization domains of the peptide portions of the fusion molecules can be used to covalently cross-link individual fusion molecules, using commercial standard cross-linking reagents (—NH2 to —NH2: Disuccinimidyl suberate (DSS) and related reagents, as described by Mattson et al., Molecular Biology Reports 17:167-183, 1993; —NH2 to —SH: (N-[e-Maleimidocaproyloxy]succinimide ester (EMCS), N-succinimidyl 3-(2-pyridyldithol)proprionate (SPDP), Succinimidyl 4-(N-maleimidomethyl)cyclohexane-1-carboxylate (SMCC), m-Maleimidobenzoyl-N-hydroxysuccinimide ester (MBS) and related reagents, according to Peeters et al., J. Immunol. Methods 120:133-143, 1989; and —SH to —SH: 1,4-bis-Maleimidyl-2,3-dihydroxybutane (BMDB) and related reagents; Pierce, Rockford, Ill.). Alternatively, two —SH groups can be directly linked through disulfide bond formation.

Preferably in these cross-linking methods, amino acid(s) are utilized that carry the desired functional groups and that are positioned at useful locations in the peptide. Alternatively, such amino acid(s) may be introduced at specified positions within the peptide. In addition, it is preferable that such functional groups be present only once per nucleic acid-protein fusion molecule, or be positioned in a way such that a higher relative reactivity in cross-link formation, compared to potential competitors, is established. This could be achieved, for example, by employing the above-described nucleic acid hybridization methods to position the desired reactive groups such that a specific cross-linking reaction is promoted.

Dynamic equilibrium state multimer formation (as described in Example 14) is advantageous to correct improper orientation or positioning.

### EXAMPLE 9

### Formation of Nucleic Acid-Protein Fusion Multimers through the Association of the Protein Portions of the Nucleic Acid-Protein Fusion Molecules

The formation of nucleic acid-protein fusion multimers can also be accomplished through the association of the multimerization domains of the protein portions of the fusion molecules. In this approach, each protein portion of a fusion molecule contains two regions, a defined multimerization domain and a compound recognition domain (for example, a randomized domain). The nucleic acid-protein fusion molecules may be generated, for example, by synthesizing the nucleic acid molecule that contains nucleic acids encoding the defined multimerization domain and the randomized compound recognition domain. Alternatively, a nucleic acid encoding the desired randomized compound recognition domain may be ligated to a selected nucleic acid sequence encoding the defined multimerization domain. Dimeric or multimeric nucleic acid-protein fusion molecules are then formed through the interaction of the defined multimerization domains. Multimerization domains may be chosen to form, for example, homodimers (e.g., GCN4 leucine zippers, as described by O'Shea et al., Science 243:538-42, 1989; and O'Shea et al., Science 254:539-44, 1991), heterodimers (e.g., Jun-Fos according to O'Shea et al., Science 245:646-8, 1989), trimers, or tetramers (Harbury et al., Science 262:1401-7, 1993; and Graddis et al., Biochemistry 32:12664-71, 1993). Alternatively, multimerization domains may also be antibody constant regions (e.g., C_{H}-C_{L}, as described by Muller et al., FEBS Lett. 422:259-64, 1998). If desired, interactions between the multimerization domains of the fusion molecules can be further strengthened by the formation of covalent disulfide bridges between the multimerization domains.

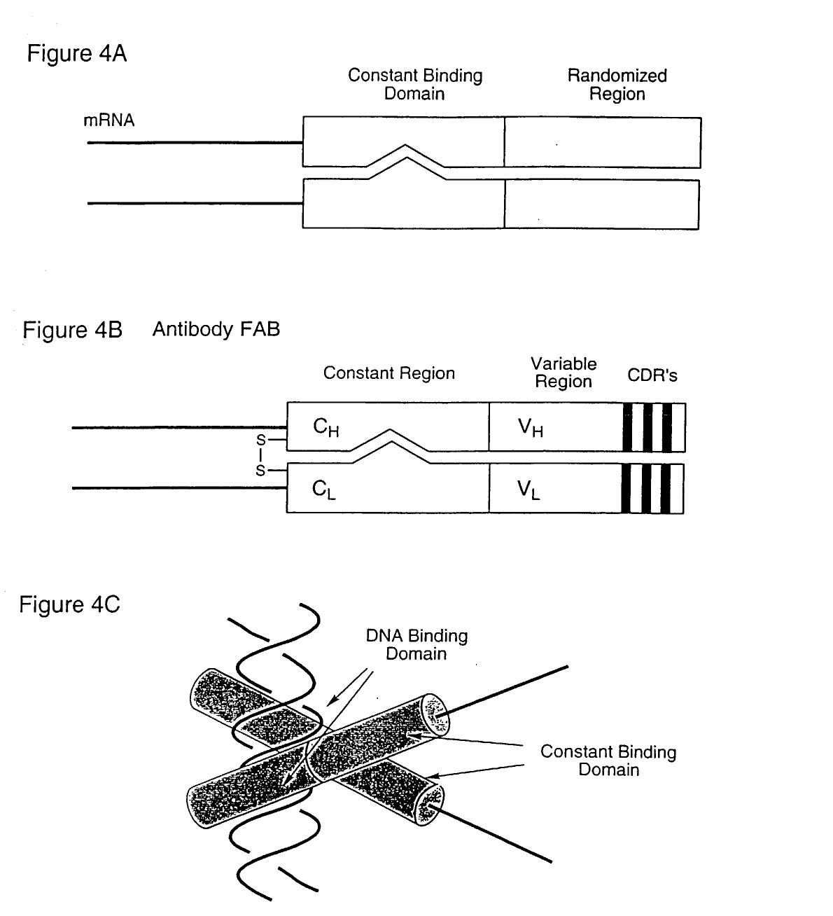

Preferably the relative orientation of the multimerization domains of each nucleic acid-protein fusion molecule are chosen such that the randomized compound recognition domains are in close proximity to each other without interfering with the nucleic acid portions of the fusion molecules. This can be achieved, for example, by introducing parallel multimerization domains (e.g., zinc finger domains, leucine zipper domains, for example, Jun-Fos leucine zipper regions, antibody C_{H}-C_{L} regions, tetrazipper regions, or other such binding domains known in the art) at the carboxy-terminus of the protein portion, with the randomized compound recognition domains at the amino-terminus (see FIGS. 4A and 4B).

### EXAMPLE 10

### Formation of Multimeric Nucleic Acid-Antibody Fab-Fragment Complexes

The formation of nucleic acid-protein fusion multimers through interaction between multimerization domains in the protein portions of the fusion molecules can be made versatile by designing those protein portions to consist of antibody Fab fragments. Dimerization is mediated through association between the constant regions, C_{H} and C_{L} (i.e., multimerization domains), followed by disulfide bond formation, allowing the randomized variable regions V_{H} and V_{L} (i.e. compound recognition domains) to be correctly positioned for recognition and binding of potential antigens. This method can be used for the construction of antibody Fab libraries (Gao et al., Proc. Natl. Acad. Sci. USA 96:6025, 1999).

### EXAMPLE 11

### Formation of Nucleic Acid-Protein Fusion Multimers for the Selection of Protein Multimers that Bind to a Desired DNA Double Helical Element

DNA binding proteins (Pomerantz et al., Biochemistry 37:965, 1998) can also be discovered by in vitro selection using nucleic acid-protein fusion multimers. As discussed above in Example 9, the C-termini of the protein portions of the fusion molecules can be fused to an appropriate dimerization domain (e.g., a leucine zipper domain or antibody constant region as shown in FIG. 4C) or nucleic acid sequence in the context of nucleic acid-protein fusion molecules. The molecule further contains a compound recognition domain that recognizes and binds DNA (e.g., a zinc finger domain). When the fusion protein dimer is contacted with a double-stranded target DNA element, the fusion proteins will recognize and bind two adjacent sequence domains on the target DNA (FIG. 4C). The use of zinc finger domains containing randomized sequences can be used to select for protein dimers that bind to a desired DNA double helical element.

### EXAMPLE 12

### Increasing Library Complexity through Assembly of Multiple Nucleic Acid-Protein Fusion Molecules

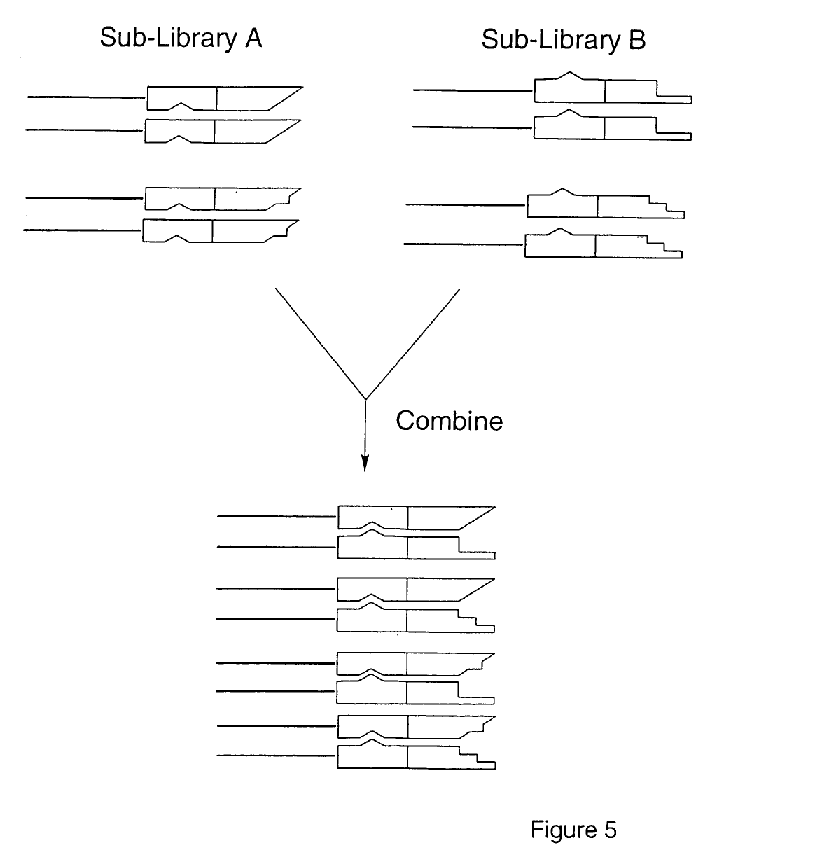

A random library of nucleic acid-protein fusion multimers can be formed by combining different libraries of nucleic acid-protein fusion molecules. For example, for dimeric constructs, the construction of a random library begins with the preparation of two independent pools of randomized template DNA (e.g., A₁₋ₙ; B₁₋ₙ). Amplification of the DNA molecules, followed by formation of nucleic acid-protein fusion molecules, for example, according to the methods of Roberts and Szostak (supra) and Szostak et al. (supra) leads to multiple copies of each member of the two pools. During the dimerization step, each particular molecule (e.g., A₁) is randomly combined with a different molecule from the other pool (e.g., Bₙ), resulting in a library of potentially unique members (A₁-B₁; A₁-B₂, A₁-B₃, . . . ) (FIG. 5). Thus, the library complexity is maximized, and virtually equals the number of possible dimeric nucleic acid-protein fusion molecule combinations that are present in the pools.

### EXAMPLE 13

### Increasing Library Complexity through Repeated Recombination during the Selection Step

Depending on the nature and strength of the forces that govern the multimerization of the nucleic acid-protein fusion molecules, dissociation and recombination steps can be introduced during the selection process, thereby continuously producing new multimers approaching the maximal molecular diversity.

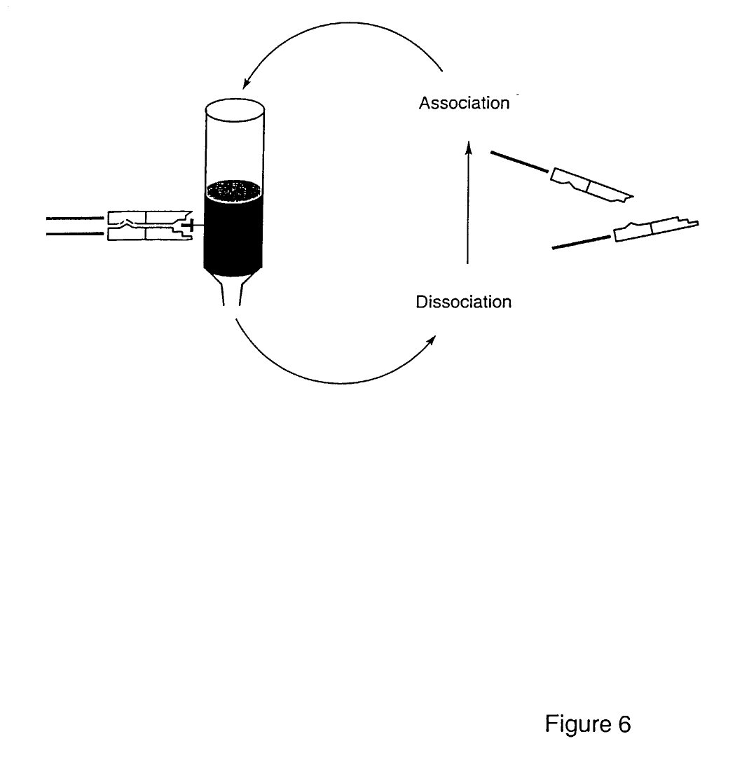

An exemplary scheme for the in vitro selection of nucleic acid-protein fusion molecules with high binding affinities for a given ligand now follows (FIG. 6). Nucleic acid-protein fusion molecules A and B are generated separately, and are subsequently multimerized using any of the methods described above. This library is then passed over a column with the desired immobilized affinity ligand, for example, a candidate compound. Those nucleic acid-protein fusion multimers that bind the ligand are retained on the column and the unbound multimeric fusions are recovered in the eluate.

In the next step, the unbound multimeric fusion complexes contained in the eluate are dissociated and recombined. This is achieved, for example, through a brief heating-cooling process for multimers that combined by nucleic acid hybridization or non-covalent protein-protein interactions. In another example, nucleic acid-protein fusion multimers held together by covalent disulfide bonds can be dissociated through reduction, and reassociated under oxidative conditions. Depending on the secondary and tertiary structure requirements, such conditions may need to allow proper refolding of the peptide domains. Once the re-multimerization takes place, the newly formed fusion complexes are applied to the affinity column described above. This process can be repeated, as desired, and where applicable, simplified by automation. When the desired number of selection rounds have been completed, the flowthrough is discarded and the selected nucleic acid-protein fusion complexes are eluted from the affinity column. The selected fusion complexes can be affinity eluted by incubating the affinity column with free ligand for an extended period of time. Alternatively, and more preferably, elution may occur by denaturation of the nucleic acid-protein fusion complexes through acid or base treatment, for example, dilute acid, as described by Roberts and Szostak (supra). The column format may be replaced by other suitable methods known in the art.

### EXAMPLE 14

### Increasing Library Diversity through Dynamic Recombination

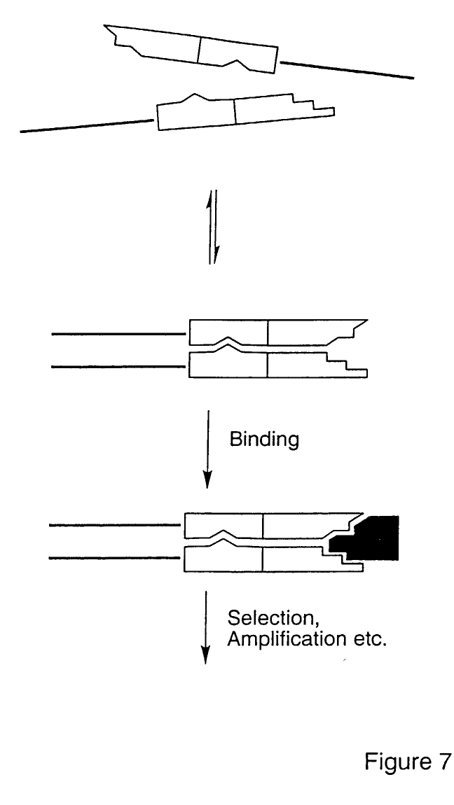

Another approach to increasing the diversity of nucleic acid-protein fusion multimers is through a modified form of the dynamic recombination described by Eliseev & Lehn, Curr Top Microbiol Immunol 243:159-72, 1999. This modified method employs individual nucleic acid-protein fusion complexes that are held together by rather weak, non-covalent interactions. In an equilibrium state, these molecules rapidly associate and dissociate, thereby constantly creating new multimeric species. Upon formation of a fusion multimer with the appropriate affinity for a ligand, the multimer binds to the ligand, resulting in an increase in the overall complex stability. By binding to the ligand, the multimer is removed from the equilibrium, and separated from those nucleic acid-protein fusion complexes that are still dissociating and recombining (FIG. 7).

A specific example of a nucleic acid-protein fusion multimer that may be used in this Example is one that is held together through nucleic acid hybridization, as described above. Depending on the length and sequence of the hybridization domain, the Tₘ (and hence the association/dissociation equilibrium state) can be adjusted to the temperature range where the selection process is performed. This results in the dissociation and recombination described above, increasing library complexity.

### EXAMPLE 15

### Repeated Generation of New Diversity through Recombination when Proceeding from one Molecular Generation to Another

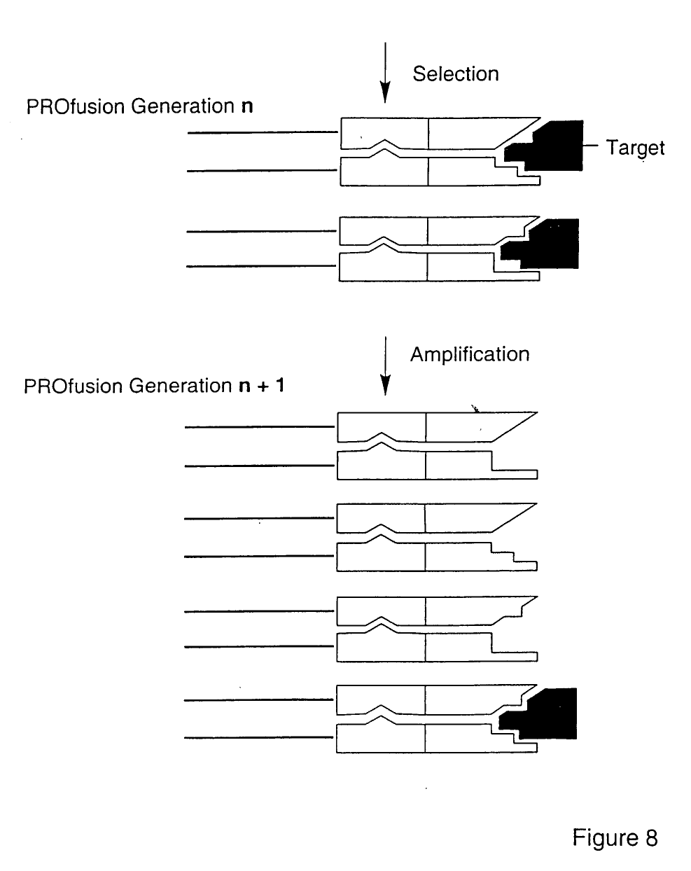

The in vitro selection steps described above yield a subset of the original pool of nucleic acid-protein fusion multimers that bind to a desired ligand. As representatives of any multimer, this is herein described for dimers (e.g., A₁-B₁; A₂-B₂). During the preparation of the next molecular generation for an additional round of selection, every selected nucleic acid-protein fusion molecule (representing a single domain A or B) is again generated in multiple copies through PCR and transcription, and added to the pool of fusion molecules to be used in the next round of selection. Thus, new diversity is generated for subsequent recombination events (e.g., A₁-B₁; A₁-B₂; A₂-B₁; A₂-B₂; FIG. 8). Repeated rounds of selection eventually lead to the enrichment of the combination of domains that optimally bind to the desired ligand.

### EXAMPLE 16

### Deconvolution of Optimal Ligand Binding Domains and Final Product Design

To continue the representative example with dimers, after several rounds of in vitro selection and selective enrichment, each of the individual pools (Aₓ, Bₓ) is reduced to a limited number of individual nucleic acid-protein fusion molecules (e.g., A_{m,n,o . . .} , B_{p,q,r . . .} ) that interact with a desired ligand. Yet, the final assignment of the correct dimerization partners (e.g., Aₙ-B_{q}) might not be obvious in all the cases. If most of the individual molecules fall into a few classes, for example, they have the same binding domain in the protein portion of the fusion, nucleic acid-protein fusion multimers can be prepared and tested selectively. If, however, the number of individual nucleic acid-protein molecules is large, any one sequence (e.g., Aₙ) can be held constant and in vitro selection with the whole population (e.g., B_{p,q,r}) can be continued for a few more rounds, to identify the appropriate partner(s) for the particular fusion molecule held constant.

Once the final assignment of dimerization partners is accomplished, it may be desirable to engineer the individual segments into one common molecule. This can be achieved, for example, by mounting the selected peptide domains onto a suitable scaffold (e.g., small organic template molecules, or peptide linkers). In addition, selected light and heavy chains of antibody Fabs can be grafted onto F_{c} fragments to generate complete antibody structures (IgG).

### EXAMPLE 17

### Rational Design of Nucleic Acid-Protein Fusion Multimers

The multimerization techniques described above can also be used for the design of multi-domain peptides or proteins with defined spatial arrangements of their subunits. This can easily be achieved through hybridization of the nucleic acid portions of the fusion molecules with other nucleic acid-protein fusion molecules or with suitable nucleic acid templates. This approach can be used to induce proximity of the multidomain peptides or proteins prior to inter-domain chemical reactions, for example, in a modification of the template-assembled synthetic proteins (TASP) methods for the creation of artificial multifunctional peptides and multi-domain receptors (Tuchscherer et al., Methods Mol. Biol. 36:261-85, 1994). This method can also be used to create antibody constructs analogous to the multivalent miniantibodies described by Plückthun and Pack (Immunotechnology 3:83, 1997).

In addition, the hybridization of nucleic acid-protein fusion molecules with nucleic acid templates does not necessarily require the RNA portion, but may also be completed with only the DNA-linker and protein portions. In such cases, the RNA can be safely removed with ribonucleases devoid of DNase activity, (e.g., RNase I, Ambion (Austin, Tex.)) prior to complex assembly.

From the foregoing description, it will be apparent that variations and modifications may be made to the invention described herein to adopt it to various usages and conditions. Such embodiments are also within the scope of the following claims.

All publications and patents mentioned in this specification are herein incorporated by reference to the same extent as if each individual publication or patent was specifically and individually indicated to be incorporated by reference.

Other embodiments are within the claims.

What is claimed is:

1. A nucleic acid-protein fusion multimer, said multimer comprising two or more fusion molecules of nucleic acid covalently bound to protein, the nucleic acid of at least one of said fusion molecules encoding the covalently bound protein, wherein said fusion molecules are hybridized to each other through complementary nucleic acid sequences.

2. A nucleic acid-protein fusion multimer, said multimer comprising two or more fusion molecules of nucleic acid covalently bound at the 3′ end to protein, wherein said fusion molecules are hybridized to each other through complementary nucleic acid sequences.

3. A nucleic acid-protein fusion multimer, said multimer comprising:
(a) two or more fusion molecules of nucleic acid covalently bound to protein, the nucleic acid of at least one of said fusion molecules encoding the covalently bound protein; and
(b) an oligonucleotide, wherein a sequence of the nucleic acid of each of said fusion molecules is hybridized to a complementary sequence of said oligonucleotide.

4. A nucleic acid-protein fusion multimer, said multimer comprising:
(a) two or more fusion molecules of nucleic acid covalently bound at the 3′ end to protein; and
(b) an oligonucleotide, wherein a sequence of the nucleic acid of each of said fusion molecules is hybridized to a complementary sequence of said oligonucleotide.

5. A nucleic acid-protein fusion multimer, said multimer comprising:
(a) two or more fusion molecules of nucleic acid covalently bound to protein; and
(b) an oligonucleotide having a bi-directional or branched structure, wherein a sequence of the nucleic acid of each of said fusion molecules is hybridized to a complementary sequence of said oligonucleotide.

6. A nucleic acid-protein fusion multimer, said multimer comprising:
(a) two or more fusion molecules of nucleic acid covalently bound to protein, wherein the nucleic acid of each of said fusion molecules comprises a polypurine tract; and
(b) an oligonucleotide comprising at least two polypyrimidine tracts, wherein said polypurine tracts of said fusion molecules are hybridized to said polypyrimidine tracts of said oligonucleotide, and wherein binding of said fusion molecules to said oligonucleotide occurs in opposite directions to form a triple helical structure.

7. The nucleic acid-protein fusion multimer of claim 6, wherein said oligonucleotide is circular.

8. The nucleic acid-protein fusion multimer of claim 6, wherein said oligonucleotide forms a clamp-like structure.

9. The nucleic acid-protein fusion multimer of claim 6, wherein said oligonucleotide comprises polyamide nucleic acids.

10. A nucleic acid-protein fusion multimer, said multimer comprising two or more fusion molecules of nucleic acid covalently bound to protein, wherein the protein portions of said fusion molecules each comprise a multimerization domain, the multimerization domains interacting through non-covalent bond formation.

11. The nucleic acid-protein fusion multimer of claim 10, wherein said multimerization domains interact to form homodimers, heterodimers, trimers, or tetramers.

12. A nucleic acid-protein fusion multimer, said multimer comprising two or more fusion molecules of nucleic acid covalently bound to protein, wherein the protein of each of said fusion molecules comprises a multimerization domain that includes a functional group, the functional group of one fusion molecule being linked to the functional group of another fusion molecule through a covalent bond.

13. The nucleic acid-protein fusion multimer of claim 12, wherein said multimerization domain comprises an antibody constant region.

14. The nucleic acid-protein fusion multimer of claim 1 or 3, wherein the protein of at least one of said fusion molecules further comprises a compound recognition domain.

15. The nucleic acid-protein fusion multimer of claim 14, wherein said compound recognition domain comprises an antibody variable region.

16. The nucleic acid-protein fusion multimer of claim 14, wherein said compound recognition domain comprises a randomized domain.

17. The nucleic acid-protein fusion multimer of claim 14, wherein said compound recognition domain interacts with DNA.

18. The nucleic acid-protein fusion multimer of claim 17, wherein said compound recognition domain comprises a zinc finger binding domain.

19. An RNA-protein fusion multimer, said multimer comprising two or more fusion molecules of RNA covalently bound to protein, wherein said fusion molecules are hybridized to each other through complementary nucleic acid sequences.

20. The RNA-protein fusion multimer of claim 19, wherein the RNA of at least one of said fusion molecules encodes the covalently bound protein.

21. The RNA-protein fusion multimer of claim 19, wherein said fusion molecules are cross-linked through cross-linking moieties positioned within the RNA of said fusion molecules.

22. The RNA-protein fusion multimer of claim 21, wherein said cross-linking moiety is psoralen.

23. A method for preparing a nucleic acid-protein multimer, said method comprising the steps of:
(a) providing two or more fusion molecules of nucleic acid covalently bound to protein, the nucleic acid of at least one of said fusion molecules encoding the covalently bound protein; and
(b) hybridizing said fusion molecules to each other through complementary nucleic acid sequences, thereby forming a nucleic acid-protein fusion multimer.

24. A method for preparing a nucleic acid-protein fusion multimer, said method comprising the steps of:
(a) providing two or more fusion molecules of nucleic acid covalently bound at the 3′ end to protein; and
(b) hybridizing said fusion molecules to each other through complementary nucleic acid sequences, thereby forming a nucleic acid-protein fusion multimer.

25. A method for preparing a nucleic acid-protein fusion multimer, said method comprising the steps of:
(a) providing two or more fusion molecules of nucleic acid covalently bound to protein, the nucleic acid of at least one of said fusion molecules encoding the covalently bound protein;
(b) providing an oligonucleotide, wherein said oligonucleotide comprises a plurality of sequences that are complementary to a partner sequence within each of said fusion molecules; and
(c) hybridizing said oligonucleotide to each of said fusion molecules, thereby forming a nucleic acid-protein fusion multimer.

26. A method for preparing a nucleic acid-protein fusion multimer, said method comprising the steps of:
(a) providing two or more fusion molecules of nucleic acid covalently bound at the 3′ end to protein;
(b) providing an oligonucleotide, wherein said oligonucleotide comprises a plurality of sequences that are complementary to a partner sequence within each of said fusion molecules; and
(c) hybridizing said oligonucleotide to each of said fusion molecules, thereby forming a nucleic acid-protein fusion multimer.

27. A method for preparing a nucleic acid-protein fusion multimer, said method comprising the steps of:
(a) providing two or more fusion molecules of nucleic acid covalently bound to protein through a peptide acceptor, wherein said fusion molecules are hybridized to each other through complementary nucleic acid sequences;
(b) providing an oligonucleotide, wherein said oligonucleotide comprises a plurality of sequences that are complementary to a partner sequence within each of said fusion molecules; and
(c) hybridizing said oligonucleotide to each of said fusion molecules, thereby forming a nucleic acid-protein fusion multimer.

28. A method for preparing a nucleic acid-protein fusion multimer, said method comprising the steps of:
(a) providing two or more fusion molecules of nucleic acid covalently bound to protein;
(b) providing an oligonucleotide having a bi-directional or branched structure, wherein said oligonucleotide comprises a plurality of sequences that are complementary to a partner sequence within each of said fusion molecules; and
(c) hybridizing said oligonucleotide to each of said fusion molecules, thereby forming a nucleic acid-protein fusion multimer.

29. A method for preparing a nucleic acid-protein fusion multimer, said method comprising the steps of:
(a) providing two or more fusion molecules of nucleic acid covalently bound to protein, wherein the nucleic acid of each of said fusion molecules comprises a polypurine tract;
(b) providing an oligonucleotide comprising at least two polypyrimidine tracts; and
(e) hybridizing said polypurine tracts to said polypyrimidine tracts, wherein binding of said fusion molecules to said oligonucleotide occurs in opposite directions to form a triple helical structure, thereby forming a nucleic acid-protein multimer.

30. A method for preparing a nucleic acid-protein fusion multimer, said method comprising the steps of:
(a) providing two or more fusion molecules of nucleic acid covalently bound to protein, wherein the protein portions of said fusion molecules each comprise a multimerization domain; and
(b) combining said fusion molecules under conditions that allow non-covalent interactions between said multimerization domains, thereby forming a nucleic acid-protein fusion multimer.

31. A method for preparing a nucleic acid-protein fusion multimer, said method comprising the steps of:
(a) providing two or more fusion molecules of nucleic acid covalently bound to protein, wherein the protein of each of said fusion molecules comprises a multimerization domain that includes a functional group; and
(b) combining said fusion molecules under conditions that allow the functional group of one fusion molecule to be linked to the functional group of another fusion molecule through a covalent bond, thereby forming a nucleic acid-protein fusion multimer.

32. The method of claim 31, wherein the covalent linkage comprises an external cross-linking agent.

33. A method for preparing an RNA-protein fusion multimer, said method comprising the steps of:
(a) providing two or more fusion molecules of RNA covalently bound to protein; and
(b) hybridizing said fusion molecules to each other through complementary nucleic acid sequences, thereby forming an RNA-protein fusion multimer.

34. A method of selecting a protein that interacts with a compound, said method comprising the steps of:
(a) providing a population of candidate nucleic acid-protein fusion multimers, said multimers comprising two or more hybridized or covalently bound fusion molecules of nucleic acid covalently bound to protein;
(b) providing a compound;
(c) contacting said compound with said population of candidate nucleic acid-protein fusion multimers under conditions that allow an interaction between said compound and said candidate nucleic acid-protein fusion multimers; and
(d) selecting a nucleic acid-protein fusion multimer that interacts with said compound, thereby selecting a protein that interacts with said compound.

35. The method of claim 34, wherein said compound is immobilized on a column.

36. The method of claim 35, further comprising the steps of:
(e) dissociating the nucleic acid-protein fusion multimers that do not interact with said compound;
(f) recombining said dissociated nucleic acid-protein fusion multimers;
(g) contacting said compound with said recombined nucleic acid-protein fusion multimers; and
(h) selecting a recombined nucleic acid-protein fusion multimer that interacts with said compound, thereby selecting a protein that interacts with said compound.

37. The method of claim 34, wherein said population, in step (a), is maintained under equilibrium conditions, whereby the individual fusion molecules of said nucleic acid-protein fusion multimers rapidly dissociate and associate with other individual fusion molecules, thereby forming new nucleic acid-protein fusion multimers.

38. The method of claim 34, further comprising the steps of:
(e) amplifying the nucleic acids of said nucleic acid-protein fusion multimers selected in step (d);
(f) generating, from said amplified nucleic acids, fusion molecules of nucleic acid covalently bound to protein;
(g) generating from those fusion molecules a second population of nucleic acid-protein fusion multimers; and
(h) repeating steps (b) through (d).

39. The method of claim 34, wherein said compound interacts with said nucleic acid-protein fusion multimer in solution and is subsequently immmobilized on a solid phase.
